(19) United States
(12) Patent Application Publication
(10) Pub. No.: US 2013/0337434 A1
(43) Pub. Date: Dec. 19, 2013

(54) Method of Preparing Magnetic Bead Type Nasopharyngeal Enzyme Immunoassay Reagents by Polymerase Chain Reaction

(71) Applicants: Ching-Yan Tsai, Keelung City (TW); Kuan-Yin Chen, New Taipei City (TW); Ping-Hung Yu, Hsinchu City (TW); Chia-Chieh Chen, Taoyuan County (TW); Chia-Ching Liu, New Taipei City (TW); Feng-Huei Lin, Taipei City (TW)

(72) Inventors: Ching-Yan Tsai, Keelung City (TW); Kuan-Yin Chen, New Taipei City (TW); Ping-Hung Yu, Hsinchu City (TW); Chia-Chieh Chen, Taoyuan County (TW); Chia-Ching Liu, New Taipei City (TW); Feng-Huei Lin, Taipei City (TW)

(73) Assignee: ATOMIC ENERGY COUNCIL-INSTITUTE OF NUCLEAR ENERGY RESEARCH, Taoyuan County (TW)

(21) Appl. No.: 13/527,212

(22) Filed: Jun. 19, 2012

(51) Int. Cl. C12Q 1/70; G01N 27/26

(52) U.S. Cl. 435/5

(57) ABSTRACT

A method of preparing magnetic bead type nasopharyngeal enzyme immunoassay reagents by polymerase chain reaction according to this invention offers in-vitro diagnostic reagents by means of utilizing nanotechnology. This method uses magnetic beads to coat EBV nuclear antigen (Epstein-Barr virus nuclear antigen, EBNA1) or early antigen (Early Antigen, EA). The use of polymerase chain reaction (polymerase Chain Reaction PCR) is for amplified detection. It is found that positive controls of different concentrations, after amplified by PCR, change in their brightness and concentration. That reveals the EBV antigens on the magnetic beads can specifically detect IgA antigens in the serum.

### BACKGROUND OF THE INVENTION

1. Field of the Invention

The present invention relates to a method of preparing magnetic bead type nasopharyngeal enzyme immunoassay reagents, especially to a method of preparing nasopharyngeal enzyme immunoassay reagents by polymerase chain reaction, in which a DNA segment is connected to two antigens as a marker and PCR amplification is applied to trace detection.

2. Description of Related Art

Epstein—Barr virus (EBV) is a herpes Branch virus which is a specific lymphocytic herpes virus of human. The genome of this virus is linear double-stranded DNA molecules. It is reported that the EBV virus-encoded RNA can be found in cells of patients with nasopharyngeal carcinoma. EBV antigens in the serum of these patients generate IgG and IgA antibodies. Therefore, researches of these antigens and antibodies are important to recognize the relationship between EBV and nasopharyngeal carcinoma and early diagnosis for nasopharyngeal carcinoma.

Roizman and de Thé G, respectively in 1982 and 1975, disclosed that nasopharyngeal cancer particularly occurs in the Chinese-speaking regions, and is widespread in the southeast of mainland China and Taiwan with the predilection of age between 30-50 years-old. Nasopharyngeal cancer is especially male-dominated, at male to female ratio of approximately 2:1. Because nasopharyngeal carcinoma is featured by non-obvious early symptoms, it is very easy to be overlooked. When the patients feel unwell and seek medical treatment, it is always found as advanced cancer which the cancer cells have spread to other organs, making the treatment very difficult. Therefore, if cancer can be found early, i.e., the cancer cells are limited around nasopharyns and treated in time, the cure rate is actually quite high (Roizman B. The herpesviruses. The New York: Plenum, 1982, p 25-103; and de Thé G; Day N; Geser A; Ho, J H; Simons M J; Sohier R; Tukei P; Vonka V Epidemiology of the Epstein-Barr virus infection and associated tumors in man. Bibl Haematol, 1975; 43: 216-20 (ISSN: 0067-7957)).

It is also disclosed that EBV virus is cultivated from African children Burkitt's lymphoma specimens by Epstein and Barr in 1964. EBV is the shape of icosahedron, and its nucleic acid is a linear double-stranded DNA. EBV is mainly transmitted through saliva, and after infection capable of producing several antigens including EBV nuclear antigen (EBNA1), early antigen (EA), membrane antigen (MA), viral capsid antigen (VCA), and lymphocyte-determined membrane antigen (LYDMA). Among them, EBNA1 is most commonly found in EBV-infection related diseases.

Nasopharyngeal carcinoma has close relationship with Epstein-Barr virus. Old and Henle et al found in 1973 that the antibody precipitated from the serum of patients with nasopharyngeal carcinoma is very similar to the Buck's lymphoma antibody. Followed by many literatures, it is proved from serological and molecular biology evidence that nasopharyngeal carcinoma has close relationship with EB virus. Deoxyribonucleic acid (DNA), ribonucleic acid (RNA) and protein of Epstein-Barr virus can be found in almost all cells in tissue biopsy specimens of the patients with nasopharyngeal carcinoma. The propagation of tumor cells comes from a single cell infected by the Epstein-Barr virus. In the later development, high concentrations of EB virus protein antibody, such as of EBNA1, EA or LYDMA, can be found in nasopharyngeal healthy individuals or in primary or recurrent patients. The patients with nasopharyngeal carcinoma will have similar IgG and IgA antibodies. Therefore, whether a EB virus antibody is detected or not can be the basis for screening the occurrence of nasopharyngeal carcinoma. (Henle G, Henle W. Epstein-Barr virus-specific of IgA serum. Antibodies, as an outstanding feature of nasopharyngeal carcinoma. Int J Cancer 1976; 17:1-7; Zong Y S, Sham J S T, Ng M H, Ou X T, Guo Y Q, Zheng S A, Liang J S, Qiu H. Immunoglobulin an against viral capsid antigen of Epstein-Barr virus and indirect mirror examination of the nasopharynx in the detection of asymptomatic nasopharyngeal carcinoma. Cancer 1992; 69:3-7; Hsu, J L, Glaser, S L. Epstein-Barr virus-associated malignancies: epidemiologic patterns and etiologic implications, Crit. Rev. Oncol Hematol 2000; 34: 27-53.). Enzyme immunoassay (Enzyme-linked immunoassay, ELISA) is very common in detection technology. There are three main categories in implementation of enzyme immunoassay: sandwich method, indirect method, and competitive method. With the characteristics of antigen-antibody binding, a sample is used with a special detection signal connected onto two antigens for detection. However, in the conventional immunoassay technology in which the EBV antigens are coated on a 96-well plate, the EBV antigens are hard to coat thoroughly on the plate due to its limited surface area, decreasing the chance of binding with the antibody. Therefore, it is difficult to effectively improve the sensitivity of the reaction, and the trace of EBV virus antigens cannot be effectively detected, failing in diagnosis of early nasopharyngeal carcinoma. The prior art cannot meet the needs for practical use.

### SUMMARY OF THE INVENTION

This invention aims at overcoming the above problems in the prior art and providing a method of preparing magnetic bead type nasopharyngeal enzyme immunoassay reagents by means of connecting a DNA segment as a marker to two antigens and application of polymerase chain reaction to amplification for trace detection.

In order to achieve the above and other objectives, a method of preparing magnetic bead type nasopharyngeal enzyme immunoassay reagents according to the present invention includes the following steps:

taking at least one nano magnetic bead treated by polyethylene glycol(PEG) and connected to EBV antigen protein to place in a 96-well plate, wherein a surface of the magnetic bead has at least one COOH functional group;

adding a test sample into the magnetic bead, wherein Anti-EBV IgA in the test sample specifically reacts with the EBV antigen on the surface of the magnetic bead in a manner to come to be adsorbed or bound onto the bead, and a magnetic field is applied to aggregate and hold them together so that those not adsorbed or bound in the test sample are separated out;

adding another Anti-Human IgA marked with a radioactive substance in the magnetic bead connecting to the Anti-EBV for further reaction, and applying a magnetic field to aggregate them together so as to separate out the Anti-Human IgA which are not adsorbed or bound, so that the marking of the magnetic bead adsorbing or binding the Anti-Human IgA can used as an indicator for signal detection; and

Immune-polymerase chain reaction (Immune PCR) detection: the Anti-Human IgA is connected to a biotin and a nucleic acid molecule is taken to connect to another biotin; the nucleic acid molecule is connected to the Anti-Human IgA through a Streptavidin; with the restriction of BamH1 to enzymes, a PCR amplification detection is carried out; and a separation process is performed to separate the nucleic acid molecule out to determine the content of antibody in the test sample.

### BRIEF DESCRIPTION OF THE DRAWINGS

FIG. 1 is a flow chart of acquiring Biotin-DNA according to the invention.

FIG. 2 is a schematic view of a reaction of EDC and Sulfo-NHS according to the invention.

FIG. 3 is a diagram showing results of non-specific experimental electrophoresis according to the invention.

### DETAILED DESCRIPTION OF THE PREFERRED EMBODIMENTS

The aforementioned illustrations and following detailed descriptions are exemplary for the purpose of further explaining the scope of the present invention. Other objectives and advantages related to the present invention will be illustrated in the subsequent descriptions and appended tables.

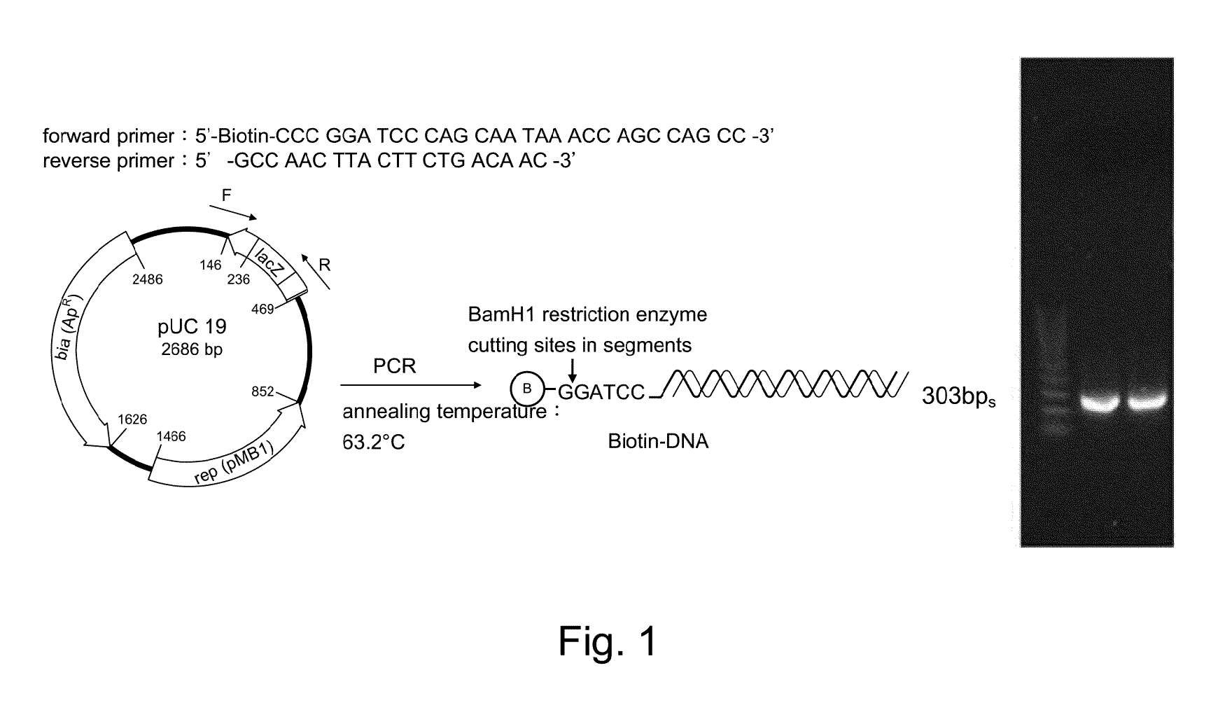

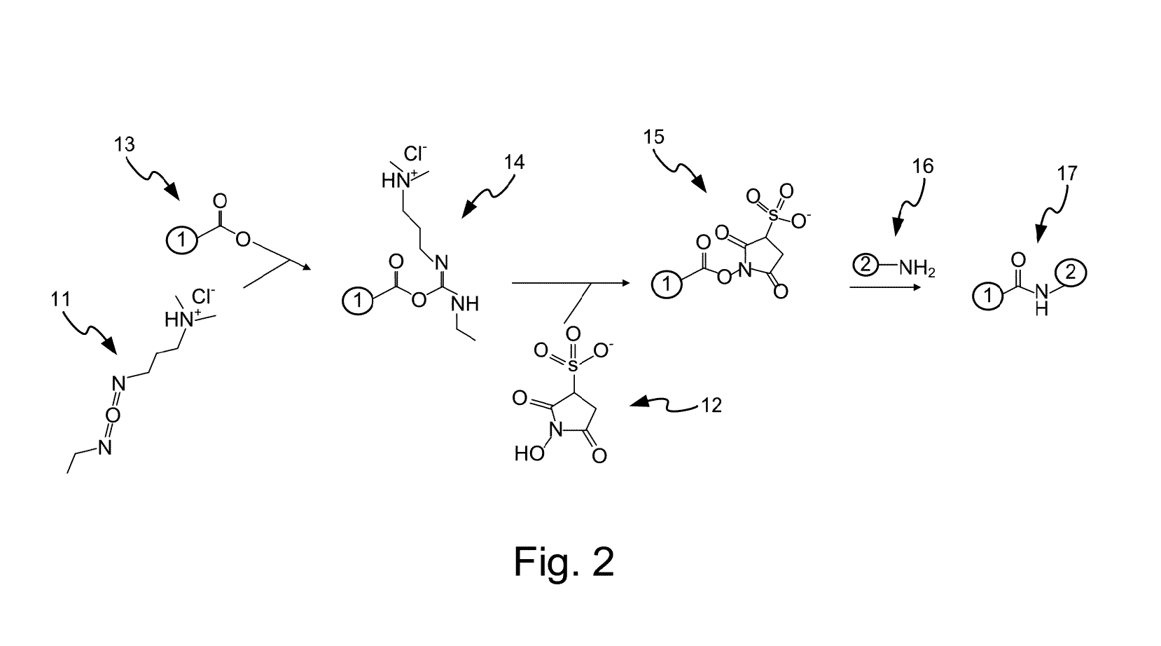

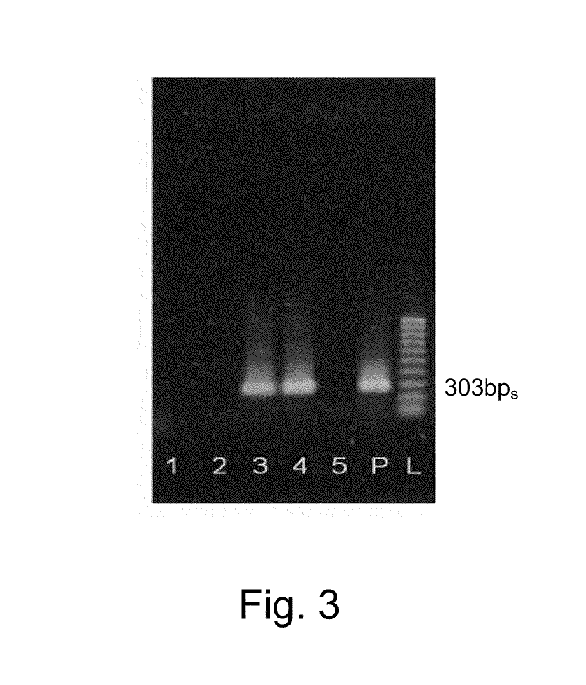

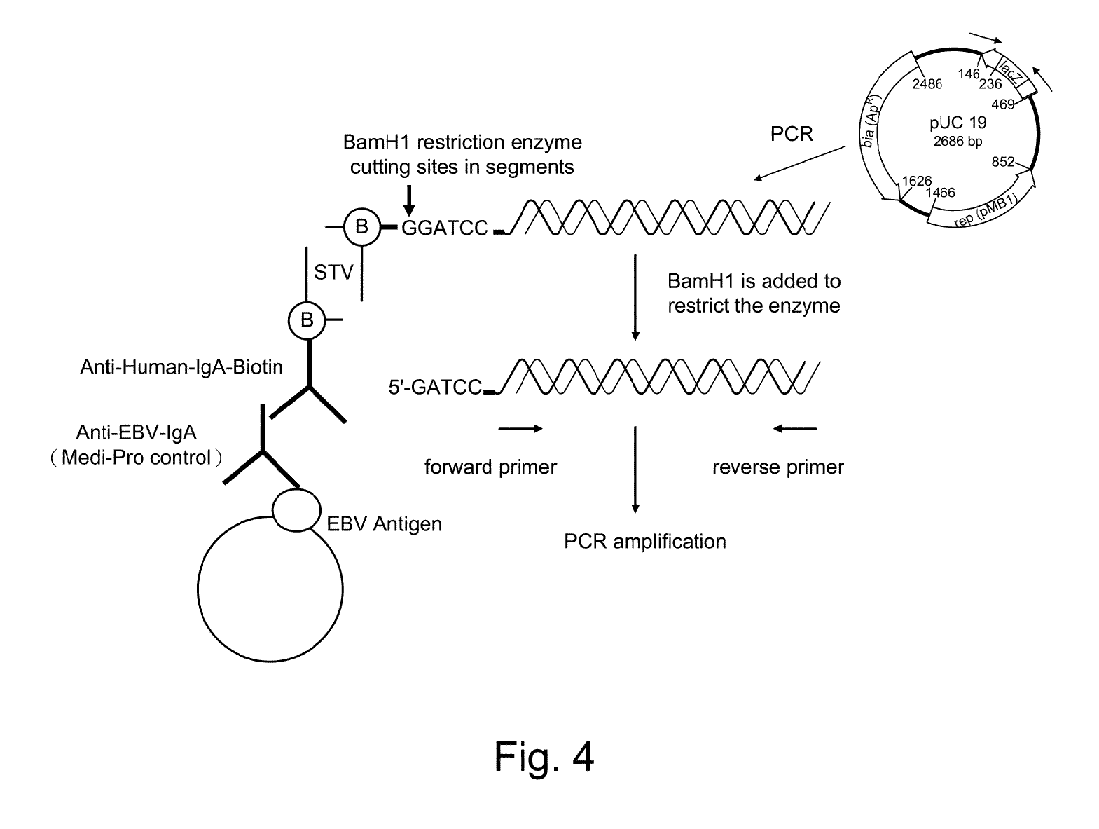

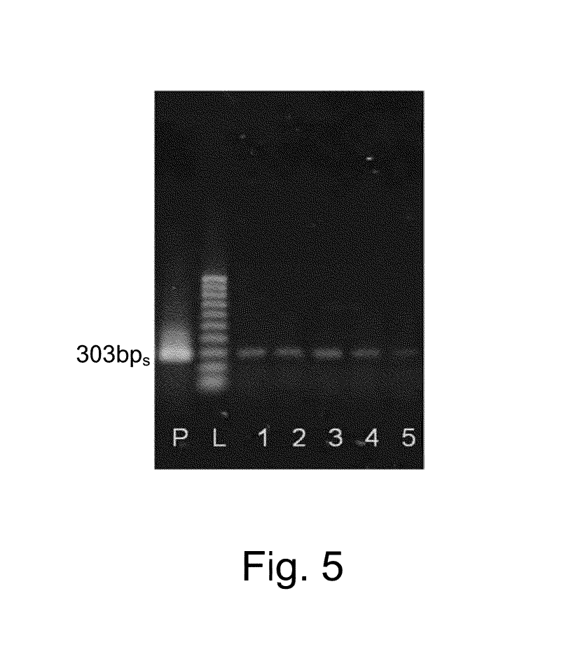

FIG. 1 is a flow chart of acquiring Biotin-DNA according to the invention. FIG. 2 is a schematic view of a reaction of EDC and Sulfo-NHS according to the invention. FIG. 3 is a diagram showing results of non-specific experimental electrophoresis according to the invention. As shown, a method of preparing magnetic bead type nasopharyngeal enzyme immunoassay reagents by polymerase chain reaction according to this invention includes at least the following steps:

(A) At least one nano magnetic bead treated by polyethylene glycol (PEG) and connected to EBV antigen protein is taken and then placed in a 96-well plate. A surface of the magnetic bead has at least one COOH functional group;

(B) A test sample is added into the magnetic bead. Anti-EBV IgA in the test sample specifically reacts with the EBV antigen on the surface of the magnetic bead in a manner to come to be adsorbed or bound onto the bead. A magnetic field is applied to aggregate and hold them together. Thereby, those not adsorbed or bound in the test sample are separated out;

(C) In the magnetic bead connecting to the Anti-EBV, another Anti-Human IgA marked with a radioactive substance is added for further reaction. Then, a magnetic field is applied to aggregate them together so as to separate out the Anti-Human IgA which are not adsorbed or bound. Thereby, the marking of the magnetic bead adsorbing or binding the Anti-Human IgA can used as an indicator for signal detection; and

(D) Immune-polymerase chain reaction (Immune PCR) detection: the Anti-Human IgA is connected to a biotin, and a nucleic acid molecule is taken to connect to another biotin; the nucleic acid molecule is connected to the Anti-Human IgA through a Streptavidin; with the restriction of BamH1 to enzymes, a PCR amplification detection is carried out; a separation process is performed to separate the nucleic acid molecule out to determine the content of antibody in the test sample. The test sample can be a serum from a nasopharyngeal patient.

In one preferred embodiment of the invention, the EBV antigen in the Step (A) can be early antigen (EA) or EBV antigen nuclear antigen (Epstein-Barr virus nuclear Antigen, EBNA1) (purchased from GeneWAy Biotech, Inc., USA), both being recombinant proteins purified from Escherichia coli (E. coli). The EA antigen contains 306˜390 amino acids of the human herpes virus HHV-4 Early Antigen C-terminal region. The EBV antigen includes amino acids of human herpes virus HHV-4 EBNA sequence No. 1-90 and sequence No. 408-498. A buffer used to store the two antigens is 50 mM of tris(hydroxymethyl) aminomethane buffer (Tris Buffer). The buffer contains a primary amine which will play a role of competitor in the follow-up experiment of coating antigen, adversely affecting the experimental results. Therefore, in the invention, the antigen is added to dialysis column purchased from Novagen in a manner of suspending in 500 ml of 0.1M, pH7.2 sodium phosphate buffer (PBS) for 12 hours of dialysis.

Test of antigen concentrations is performed by using Micro-BCA Kit (Pierce Biotechnology, USA). 2.0 mg/ml of bovine serum albumin (BSA) standard solution within the Micro-BC Kit is taken and diluted in accordance with predetermined proportions to be standard solutions of 0, 0.5, 1, 2.5, 5, 10, 20 and 40 μg/ml. Trace amount of reagent A (MA) solution, trace amount of reagent (B) (MB) solution and trace amount of reagent C(MC) solution within the Miro-BCA Kit are used to prepare a BCA working solution which is mixed with the standard solutions of all concentrations and dialyzed EBV antigen in equal volume, and then added in the 96-well plate for observation of OD562 absorbance values.

In addition, in the invention, pU19 plasmid nucleic acid (Plasmid DNA) is used as a PCR template, and DNA polymerase is used for PCR amplification. In operation, the following reagents are added in turns and respectively subject to PCR reaction after mixed thoroughly: 1 μg of pUC 19 Plasmid DNA, 1 μl and 10 Mm of dNTP, 5 μl and 10× of TAG Buffer, 1 μl and 30 μM of forward primer, with water up to a total volume of 50 μl; 5′-Biotin-CCC GGA TCC CAG CAA TAA ACC AGC CAG CC-3′, 1 μl and 30 μM reverse primer, with water up to a total volume of 50 μl; 5′-GCC AAC TTA CTT CTG ACA AC-3′, and 1 μl of TAG DNA Polymerase, with water up to a total volume of 50 μl. The PCR reaction is performed at the following conditions for denature: heating up to 97° C. in 30 seconds; annealing at a annealing temperature which is set according to the designed primer and calculated from the formula Tm=(A+T)×2+(C+G)×4, wherein the annealing temperature is in the range of Tm±10° C.; and elongation by cooling to 72° C., wherein the period of time for cooling is based on the length of a selected segment. For example, it takes about 60 seconds for synthesis of 1k of the base. The reaction stops after 30 cycles. QIAquick® PCR Purification Kit is subsequently used to purify the reaction products: Five times in volume of buffer (Buffer PB) is added to 1 time in volume of the PCR products; after mixing thoroughly, the mixture is added into a spin column; the spin column is operated at 12,000 rpm for 30 to 60 seconds to remove the filtrate; 0.75 ml of buffer (Buffer PE) is added and then centrifuged at 12,000 rpm for 30 to 60 seconds to remove the filtrate, and further centrifuge for 1 minute more to remove the residue of the filtrate; the centrifuge column is replaced with a new 1.5 ml tube; 50 μl of buffer (Buffer EB) or sterile water (DEPC—H₂O) is added to a centrifugal chromatography column and stays at room temperature for 1 minute; and the centrifuge column is operated at 14,000 rpm for 1 minute to obtain a purified PCR product of 303 bps in length. This PCR product has Biotin at 5′ terminal and contains BamH1 restriction enzyme cutting sites in segments (as shown in FIG. 1).

At the Step (A) in one preferred embodiment, in order to avoid any non-specific binding of DNA, the magnetic bead is connected to the PEG protein in advance before connected to the antigen. The. PEG protein is one kind of surfactants, and contains a hydrophilic terminal which makes itself water-soluble. Furthermore, it carries negative charges in liquid, acting like waving seaweed, which effectively avoids the non-specific binding of DNA. In connecting the PEG protein to the magnetic bead, 1-ethyl-(3-dimethylamino-propyl) carbodiimide hydrochloride (EDC) and N-hydroxy thio succinimide (Sulfo-NHS) are used for experiment. As shown in FIG. 2, 50 μl of EDC11, 50 μl of Sulfo-NHS (purchased from of Thermo®) 12 and the magnetic bead 13 are added and subject to reaction at room temperature for 30 min. A carboxyl functional group (COOH functional group) on the surface of the magnetic bead comes to react with EDC11 first to form an unstable intermediate product 14, and then combine with Sulfo-NHS12 to form a stable product 15. PEG16 is added for further reaction at room temperature for one hour. An amino group (NH2) on the PEG protein combines with the carboxyl group on the magnetic bead to form a stable amide bond 17. A superblocking buffer is added for blocking reaction for two hours at room temperature. The experiment is therefore completed. The magnetic bead connecting to PEG is placed at 4° C. for preservation.

In order to determine the performance of PEG to facilitate follow-up operation of Immuno-PCR experiments, DNA non-specific experimental test is performed. First, 10 μl of the magnetic bead connecting to PEG is placed in the 96-well plate. Pure water is added to dilute 5000 times 100 u1 of Bio-DNA segments. A negative control is subject to reaction after pure water is added twice. A positive control is prepared by using pUC19 as a template for PCR to determine the size of DNA. After a shock reaction is performed at 37° C. for 15 minutes and washed 10 times, BamH1 is added to restrict the enzyme. Further reaction is performed at 37° C. for 15 minutes. A magnet is used to attract the magnetic bead to be against a bottom of the 96-well plate. A supernatant is then taken for PCR amplification. A conventional gel electrophoresis and Exprion® automatic electrophoresis equipment are used for concentration test. The test results are shown in FIG. 3 and Table 1. In FIG. 3, Groups 1 and 2 represent PEG-treated magnetic beads; Groups 3 and 4 represent untreated magnetic beads; Group 5 represents the negative control; and Group P represents the positive control.

TABLE 1

| Groups | Concentration (ng/ul) |
|---|---|
| PEG-treated magnetic bead | 0.0 |
| PEG-treated magnetic bead | 0.0 |
| Untreated magnetic bead | 19.6 |
| Untreated magnetic bead | 18.7 |
| Negative control | 0.0 |
| Positive control | 28.4 |

The magnetic bead after coated by PEG is subject to non-specific test. It is found that no signals are detected from the PEG-treated magnetic beads after PCR amplification. In contrast, the untreated magnetic beads non-specifically connecting to residual DNA segments and therefore signals are detected after PCR amplification.

Subsequently, the EBV antigen is connected to the PEG-treated magnetic bead. The process of connecting the EBV antigen to the PEG-treated magnetic bead is the same as the above one.

50 μl of EDC and 50 μl of Sulfo-NHS are added to react with the magnetic bead at room temperature for 30 minutes. The carboxyl functional groups on the surface of the magnetic bead comes to react with EDC in advance to form an unstable intermediate product which then combines with Sulfo-NHS to form a stable product. At this moment, the carboxyl on PEG is activated. Then, the EBV antigen is added for further reaction at room temperature for one hour. The amino on the antigenic protein combines with the carboxyl group on PEG to form a stable amide bond. Finally, the superblocking buffer is added for blocking reaction at room temperature for two hours. The experiment is thereby completed. Finally the magnetic bead connecting to the antigen stays at 4° C. for preservation.

FIG. 4 is a schematic flow chart of immune-PCR process according to the invention. FIG. 5 is a diagram showing results of electrophoresis for Immuno-PCR experiments. As shown, when Immuno-PCR experiments are performed, 5 μl of PEG-treated magnetic bead connecting to EA or EBNA1 antigen protein is placed in a 96-well plate. In this embodiment, EBNA1 antigen protein is used. Then, 100 μl of Medio ELISA Kit positive control A which is diluted according to predetermined concentration gradients is added. The concentration gradients are 10⁻¹,10⁻³,10⁻⁶ and 10⁻⁹, respectively. The negative control can be substituted with a superblocking buffer. Furthermore, pUC19 is used as the template for PCR to confirm the DNA size, which is referred as the positive control. After the reaction has been carried out for 1 hour at 37° C., one time of washing buffer PBS (Washing Buffer in PBS, purchased from CYNDOR) is poured into a BioTekR ELx405 ELISA washing machine for cleaning. One powerful magnet is placed under this ELISA machine in order to attract the magnetic bead at a bottom of the plate for cleaning. Thereby, a great number of samples can be treated in one batch, effectively enhancing the efficiency of the experiments. GOAT ANTI HUMAN IgA: Biotin (purchased from Serotec) is diluted with the superblocking buffer at ratio of 1:1000 to wash six times after being shocked at 37° C. for 30 minutes. A chain anti-biological protein is diluted with the superblocking buffer to be 1000 times in volume. 100 μl of diluted chain anti-biological protein is taken to wash 10 times after being shocked at room temperature for 30 minutes. Bio-DNA is diluted to be 5,000 times in volume. 50 μl of the diluted Bio-DNA is taken to wash 10 times after being shocked at room temperature for 15 minutes. BamH1 is added to restrict the enzyme. BamH1 is subject to reaction at 37° C. for 15 minutes and then the magnetic bead is adsorbed on the bottom of the 96-well plate by means of the magnet. The supernatant is taken for detecting the PCR amplification. Gel electrophoresis and Exprion® PCR instrument are used to observe the brightness and concentration. The results are shown in FIG. 5 and Table 2, in which Group 1 represents the positive control A (10⁻¹); Group 2 represents the positive control A (10⁻³); Group 3 represents the positive control A (10⁻⁶); Group 4 represents the positive control A (10⁻⁹); Group 5 represents the negative control; and Group P stands for the positive control.

TABLE 2

| Group | Concentration (ng/μl) |
|---|---|
| 1. positive control A (10⁻¹) | 0.7 |
| 2. positive reference substance A (10⁻³) | 0.5 |
| 3. positive control (10⁻⁶) | 0.5 |
| 4. positive control (10⁻⁹) | 0.4 |
| 5. negative control | 0.1 |
| P. positive control | 32.3 |

These results reveal that even though the positive control A is diluted to be 10⁻⁹, the signal can still be detected for amplification. In comparison with the colorimetric method, concentration of 10⁻³ is the upper limit to be detected. Nearly 6 orders in concentration can be reached in detection, which means Immuno-PCR does have a relatively high sensitivity.

In this method, the present invention has made great improvement to the conventional immunoassay techniques. With the use of the EBV antigen-connected magnetic bead as medium, the nano magnetic bead which has spherical particles is very homogeneous, good suspension and greater surface area than the ELISA plate is capable of capturing more antigens, making the experiments more sensitive. After the EBV antigens of the same concentrations are coated on the magnetic bead and the colorimetric experiment is carried out on the 96-well ELISA plate, the obtained OD450 absorbance value of the magnetic bead reads higher. It reveals that the EBV antigens do be connected to the magnetic bead, more antigens are captured and reaction sensitivity is magnificently improved.

It is confirmed that the magnetic bead is stable and homogeneous for experiments. In addition to Immuno-PCR, it is also ready for cold light luminescence test because the sensitivity of cold light can reach up to 5 orders in detection, the standard of nasopharyngeal carcinoma detection. Furthermore, it is less likely to encounter problems of non-specific binding. Experimentally, it may be more convenient and fast. Therefore, the technology of this invention can be regarded as a variant of the sandwich method, which uses a serum antibody of a patient as a sample for detection. The types of detection signals (Detection Signal) are quite a bit. For instance, the sample can be connected to horseradish peroxidase (HRP) as a substrate, and then subject to tetramethylbenzidine (TMB) colorimetric method or cold light method, or marked with a radioactive marker for detection. The implementation of the detection according to the invention mainly includes connecting a DNA segment as a marker onto two antigens and performing PCR amplification for trace detection.

This invention utilizes the principle of nanotechnology to develop a reagent for in-vitro diagnostics for diagnosing and treating early nasopharyngeal carcinoma patients, increasing the probability of cure. This invention has made effort in improving the conventional ELISA detection method. For the medium used to coat the EBV antigen, the magnetic bead which is Dynabeads MyOne Carboxylic Acid bead in this embodiment is in the shape of sphere which has larger surface area than conventional 96-well plate shape. Therefore, it can coat more antigens at one time, increasing the sensitivity up to 500 times and therefore significantly enhancing the opportunity of binding with the antigens. Additionally, the magnetic property grants more convenience to the operation of experiment. For the method detection, the typical ELISA colorimetric method is substitute with Immuno-PCR method. The inherently high sensitivity and amplification effect of PCR contributes to detect a trace of the EBV, furthermore significantly increasing the detection sensitivity. Therefore, this invention can be used to diagnose the occurrence of early nasopharyngeal carcinoma. In light of above, the method of preparing magnetic bead type nasopharyngeal enzyme immunoassay reagents by polymerase chain reaction according to this invention can effectively overcome the shortages in the prior art by utilizing the nanotechnology technology and has successfully develop in-vitro diagnostic reagents. The magnetic bead is used to coat the EBV nuclear antigens Epstein-Barr virus nuclear Antigen, EBNA1) or early antigen (Early Antigen, EA). The polymerase chain reaction (PCR) is used for amplification by detection. It is found that the positive controls of different concentrations after PCR amplification are changed in brightness and concentration. That reveals the EBV antigens on the magnetic beads can specifically detect IgA antigens in the serum. The polymerase chain reaction amplification can be applied to the detection of trace of antibody, saving the experimental time and enhancing the stability of experiment.

The descriptions illustrated supra set forth simply the preferred embodiments of the present invention; however, the characteristics of the present invention are by no means restricted thereto. All changes, alternations, or modifications conveniently considered by those skilled in the art are deemed to be encompassed within the scope of the present invention delineated by the following claims.

1. A method of preparing magnetic bead type nasopharyngeal enzyme immunoassay reagents for polymerase chain reaction, comprising at least the following steps:
(A) placing at least one nano magnetic bead treated with polyethylene glycol (PEG) and connected to at least one EBV antigen protein in a 96-well plate, wherein the surface of the magnetic bead has at least one COOH functional group;
(B) adding a human test sample to the well, wherein Anti-EBV IgA in the test sample specifically binds to the EBV antigen on the surface of the magnetic bead creating an aggregate, and applying a magnetic field to the aggregate and washing out unbound Anti-EBV IgA;
(C) adding an Anti-Human IgA marked with a substance to the well, and applying a magnetic field to wash out the unbound Anti-Human IgA; and
(D) adding a nucleic acid molecule connected to biotin and having a restriction site between the biotin and the nucleic acid molecule adding Streptavidin to the well; washing unbound nucleic acid out of the well; removing the bound nucleic acid with a restriction enzyme; PCR amplifying the restricted nucleic acid; and performing a separation process to separate the nucleic acid molecule out to determine the presence of antibody in the test sample.

2. The method of claim 1, wherein the EBV antigen is early antigen (EA) or EBV antigen nuclear antigen (Epstein-Barr virus nuclear Antigen, EBNA1).

3. The method of claim 2, wherein the EBNA1 antigen includes both human herpes virus HHV-4 EBNA amino acids 1-90 and 408-498.

4. The method of claim 2, wherein the EA antigen comprises 306˜390 amino acids of the human herpes virus HHV-4 Early Antigen C-terminal region.

5. The method of claim 1, wherein at Step (A), the magnetic bead is connected to the PEG before the magnetic bead is connected to the antigen, by means of adding 1-ethyl-(3-dimethylamino-propyl)carbodiimide hydrochloride (EDC) and N-hydroxy thio succinimide (Sulfo-NHS) to react with the magnetic bead at room temperature, wherein a carboxyl functional group on a surface of the magnetic bead comes to react with EDC first to form an unstable intermediate product which then combines with Sulfo-NHS to form a stable product; and adding PEG for further reaction at room temperature, wherein an amino group (NH₂) on the PEG combines with the carboxyl group on the magnetic bead to form a stable amide bond.

6. The method of claim 1, wherein at the Step (A) the magnetic bead is connected to the EBV antigen after connection of PEG to the bead, by means of adding EDC and Sulfo-NHS to react with the magnetic bead connected to PEG at room temperature, wherein a carboxyl functional group on a surface of the magnetic bead comes to react with EDC first to form an unstable intermediate product which then combines with Sulfo-NHS to form a stable product; the carboxyl on PEG is activated; the EBV antigen is added for further reaction at room temperature; and amino (NH₂) on the antigenic protein combines with the carboxyl group on PEG to form a stable amide bond.

7. The method of claim 1, wherein the test sample is a serum from a patient having nasopharyngeal carcinoma.

8. The method of claim 1, wherein the separation process at the Step (D) is gel electrophoresis.

9. The method of claim 1, wherein the substance is a radioactive marker and the test for binding of the anti-human IgA is to detect radioactivity.

10. The method of claim 1, further comprising determining the amount of antibody in a test sample.

11. The method of claim 1, wherein the primers having SEQ ID Nos: 1 and 2 are used for the PCR.

12. The method of claim 1, wherein the restriction enzyme is BamH1.


SEQUENCE LISTING (2 sequences)

SEQ ID NO: 1 - DNA, 29 bp, artificial sequence
  Forward primer
cccggatcccagcaataaaccagccagcc

SEQ ID NO: 2 - DNA, 20 bp, Artificial sequence
  Reverse primer
gccaacttacttctgacaac